(19) United States
(12) Patent Application Publication
(10) Pub. No.: US 2006/0271154 A1
(43) Pub. Date: Nov. 30, 2006

(54) Methods and systems for treating vulnerable plaque

(75) Inventor: Marvin L. Woodall, Doylestown, PA (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 11/439,236

(22) Filed: May 24, 2006

Related U.S. Application Data
(60) Provisional application No. 60/684,171, filed on May 25, 2005.

(51) Int. Cl. A61F 2/06

(52) U.S. Cl. 623/1.11

(57) ABSTRACT

The invention provides methods and catheter-based delivery systems for treating vulnerable plaque atherosclerotic conditions using lightweight vulnerable plaque shields. Combination catheters are provided that include a selectively deployable occlusion balloon to occlude blood flow and a mechanism for selectively deploying a lightweight vulnerable plaque shield, which is either self-expanding or balloon-deployable, within a blood vessel.

### FIELD OF THE INVENTION

The invention relates to the field of diagnosis and treatment of atherosclerotic lesions.

### BACKGROUND OF THE INVENTION

Coronary artery disease (CAD) is a major cause of death, disability, and healthcare expense. Until recently, it was widely thought that the predominant cause of CAD is a progressive increase of hard plaque in the coronary arteries. The atherosclerotic disease process of hard plaques leads to a critical narrowing (stenosis) of the affected coronary artery and, as blood flow is occluded, produces angina. The progressive narrowing of the artery can eventually trigger the formation of a blood clot that causes an abrupt cardiac ischemia, i.e., heart attack, by choking off the flow of oxygen rich blood to the heart muscle. A dislodged clot may also travel to and lodge in a blood vessel of another organ such as the brain, resulting in a thrombotic stroke.

A current paradigm for treating stenotic atherosclerotic plaques is angioplasty and stenting, in which the stenosis is opened by intraluminal expansion of a high-pressure angioplasty balloon, concomitantly with, or followed by, the placement of a strong, metallic stent having sufficient radial strength to maintain the artery in an open state.

More recent clinical data suggests that the majority of heart attacks result from the rupture of vulnerable plaques rather than hard plaques. In many instances, vulnerable plaques do not impinge on the vessel lumen but, instead, are embedded in the wall of an artery. Effective detection and treatment of vulnerable plaques is complicated since a patient typically does not experience angina and since conventional angiography or fluoroscopy techniques are not well suited for detecting such plaques.

Vulnerable plaques have characteristic physical, chemical and biological signatures. The majority of vulnerable plaques include a lipid pool, a necrotic ring, and a dense infiltrate of macrophages contained by a thin fibrous cap, generally having a thickness of 50 or fewer microns (micrometers). Some fibrous caps may even have a thickness of around 2 microns.

The present invention addresses the need for interventional techniques and systems for treating vulnerable plaques.

### SUMMARY OF THE INVENTION

One aspect of the invention provides a method for treating a vulnerable plaque condition in an animal or human patient, that includes the steps of: determining the location of a vulnerable plaque in a patient; advancing a guide wire through the vasculature of the patient to the location of the vulnerable plaque; advancing a combination occlusion and shield deployment catheter over, i.e., along, the guide wire to the location of the vulnerable plaque, the catheter including: an occlusion balloon, and an unexpanded, self-expanding vulnerable plaque shield, the shield being located proximally along the catheter with respect to the occlusion balloon; deploying the occlusion balloon to occlude blood flow; and, while blood flow is at least substantially occluded by the deployed occlusion balloon, deploying the self-expanding vulnerable plaque shield so that it at least partially, and preferably, at least substantially, covers the vulnerable plaque.

A related aspect of the invention provides a combination occlusion and shield deployment catheter for treating vulnerable plaque conditions, that includes: a distal end for insertion into a patient's body and a proximal end; a selectively deployable occlusion balloon capable of occluding blood flow in a blood vessel; and a selectively deployable vulnerable plaque shield, located proximally along the catheter with respect to the occlusion balloon. The catheter may also include one or more structural elements or features for guiding the catheter over or along a guide wire.

Another aspect of the invention provides a method for treating a vulnerable plaque condition in an animal or human patient, that includes the steps of: determining the location of a vulnerable plaque in a patient; advancing a guide wire through the vasculature of the patient to the location of the vulnerable plaque; advancing a combination occlusion and shield deployment catheter over/along the guide wire to the location of the vulnerable plaque, the catheter including an occlusion balloon for occluding blood flow and an unexpanded, balloon-expandable vulnerable plaque shield mounted circumferentially about a deployment balloon, the shield being located proximally along the catheter with respect to the occlusion balloon; deploying the occlusion balloon to occlude blood flow; and while blood flow is at least substantially occluded by the deployed occlusion balloon, deploying the vulnerable plaque shield by expanding the deployment balloon so that the shield at least partially, and preferably, at least substantially, covers the vulnerable plaque.

A related aspect of the invention provides a combination occlusion and shield deployment catheter for treating vulnerable plaque conditions, that includes: a distal end for insertion into a patient's body and a proximal end; a selectively deployable occlusion balloon capable of occluding blood flow in a blood vessel; a selectively balloon-expandable vulnerable plaque shield mounted circumferentially about a deployment balloon, the shield being located proximally along the catheter with respect to the occlusion balloon. The catheter may also include one or more structural elements or features for guiding the catheter over or along a guide wire.

Still another aspect of the invention provides a vulnerable plaque shield having pore sizes that promote endothelialization. A related aspect of the invention provides an at least partially filament-based vulnerable plaque shield, for example, an at least substantially tubular prosthesis formed, for example, by braiding, weaving and/or knitting metallic and/or non-metallic filaments.

### BRIEF DESCRIPTION OF THE DRAWINGS

FIG. 1A shows, in cross-section, an example of a vulnerable plaque shield delivery catheter according to the invention.

FIG. 1B shows a step in the operation of the delivery catheter of FIG. 1A.

FIG. 1C shows a further step in the operation of the delivery catheter of FIG. 1A.

FIG. 1D shows a still further step in the operation of the delivery catheter of FIG. 1A.

### DETAILED DESCRIPTION OF THE INVENTION

The invention is generally directed to methods and systems for treating vulnerable plaque conditions of blood vessels.

Since vulnerable plaques are not typically associated with significant stenosis, it is not necessary to use a shield that is a radially strong device like the stents typically used for treating stenotic, hard atherosclerotic lesions. Instead, the primary requirements and function of the vulnerable plaque shield are that it remains in place in contact with the vulnerable plaque region of the vessel wall and that it stabilizes and/or isolates the vulnerable plaque to prevent or reduce the risk that it will rupture into the vessel lumen. Conventional stents used to treat stenosis tend to maximize the percent open area of the stent wall in order to minimize the amount of metal in the artery, which has been believed to reduce the likelihood of thrombosis. Further, conventional stents with their large cell sizes (of the interstitial spaces in the stent wall) and high radial strengths are known to sometimes cut into a stenotic plaque upon expansion so that plaque material projects into the lumen of the stent. In contrast, the vulnerable plaque shields of the present invention are characterized by a small cell size that serves to more effectively support and protect a vulnerable plaque region from rupture while also more effectively promoting endothelialization of the lumen-side wall of the implanted shield prosthesis. The terms “cell size” and “pore size” refer to the size of the empty areas in the wall of a prosthesis that are, for example, bounded by the braided wires of a braided shield prosthesis, formed by machining of a solid tubular structure, or otherwise formed in the wall of a shield prosthesis. As used herein, cell sizes are measured in area and pore sizes are measured in average diameter.

In one embodiment of the invention, the cell sizes of the shield wall are at least predominantly in the range of 0.038 mm²-0.180 mm². In another embodiment of the invention, the average cell size of the shield wall is in the range of about 0.038 mm² to about 0.180 mm². In one variation of the embodiments, the shield is an at least substantially braided structure, for example, one formed from about 48 to about 144 wires. In another variation, the wires are at least predominantly from about 12 to about 50 microns thick or from about 0.0005 to about 0.002 inches thick. In a further variation, the wires are, at least predominantly, from about 25 to about 50 microns thick or from about 0.001 to about 0.002 inches thick. In still another variation, the braid angle can be up to about 105 degrees. In one embodiment of the invention, the shield is an at least substantially braided structure of about 48 to about 60 wires with a percent open area in the shield wall of about 70% to about 86%. In another embodiment, the shield is an at least substantially braided structure of about 48 to about 60 wires having an average cell size of about 0.180 mm².

In contrast to conventional stents, the small pore sizes of the vulnerable plaque shields of the present invention more effectively stabilize the vulnerable plaque lesion, while at the same time more effectively promoting endothelialization of the inner lumen surface. In one embodiment of the invention, the pore sizes of the shield wall are at least predominantly less than 500 microns. In another embodiment of the invention, the pore sizes of the shield wall are at least predominantly less than 400 microns. In a further embodiment of the invention, the pore sizes of the shield wall are at least predominantly less than 300 microns. In still another embodiment of the invention, the pore sizes of the shield wall are at least predominantly less than 200 microns. In one embodiment of the invention, the pore sizes of the shield wall are at least predominantly less than 100 microns. In another embodiment of the invention, the pore sizes of the shield wall are at least predominantly less than 75 microns. In a further embodiment, the pore sizes of the shield wall are at least predominantly less than 50 microns. In another embodiment, the pore sizes of the shield wall are at least predominantly less than 25 microns. In a further embodiment, the pore sizes of the shield wall are at least predominantly in the range of about 3 microns to about 100 microns. In another embodiment, the pore sizes of the shield wall are at least predominantly in the range of about 3 to about 75 microns. In another embodiment, the pore sizes of the shield wall are at least predominantly in the range of about 3 to about 50 microns. In another embodiment, the pore sizes of the shield wall are at least predominantly in the range of about 3 to about 40 microns. In another embodiment, the pore sizes of the shield wall are at least predominantly in the range of about 3 to about 30 microns. In another embodiment, the pore sizes of the shield wall are at least predominantly in the range of about 5 to about 30 microns. In another embodiment, the pore sizes of the shield wall are at least predominantly in the range of about 5 to about 25 microns. In another embodiment, the pore sizes of the shield wall are at least predominantly in the range of about 10 to about 20 microns. In another embodiment, the pore sizes of the shield wall are at least predominantly in the range of about 20 to about 40 microns. In another embodiment, the pore sizes of the shield wall are at least predominantly in the range of about 20 to about 50 microns.

Thus, according to one embodiment of the invention, a self-expanding shield with characteristics for better containing vulnerable plaque and promoting endothelialization, can be used to treat a vulnerable plaque condition. In another embodiment, a balloon-deployable shield with characteristics for better containing vulnerable plaque and promoting endothelialization, can be used to treat a vulnerable plaque condition. In one variation of this embodiment, the shield is deployable by a low-pressure balloon, such as a low-pressure elastomeric balloon, rather than a high-pressure balloon as required for a conventional balloon-expandable stent used to treat stenotic atherosclerotic lesions. Balloon pressure for a conventional high-pressure angioplasty balloon or stent delivery balloon is 12 to 16 ATMS or 180 to 240 psi. Those skilled in the art will appreciate that materials can be readily selected by type, thickness and/or gauge to provide balloon-expandable, tubular endoprostheses that are expandable by low pressure.

One embodiment of the invention provides a method for treating a vulnerable plaque condition in an animal or human patient, that includes the steps of: determining the location of a vulnerable plaque in a patient; advancing a guide wire through the vasculature of the patient to the location of the vulnerable plaque; advancing a combination occlusion and shield deployment catheter over, i.e., along, the guide wire to the location of the vulnerable plaque, the catheter including: an occlusion balloon; and an unexpanded, self-expanding vulnerable plaque shield, the shield being located proximally along the catheter with respect to the occlusion balloon; deploying the occlusion balloon to occlude blood flow; and, while blood flow is at least substantially occluded by the deployed occlusion balloon, deploying the self-expanding vulnerable plaque shield so that it at least partially, and preferably, at least substantially, covers the vulnerable plaque.

The steps of determining the location of a vulnerable plaque in a patient and advancing a guide wire through the vasculature of the patient to the location of the vulnerable plaque can be performed concomitantly, for example, when a diagnostic catheter preloaded with a guide wire is used to determine the location of a vulnerable plaque, or they may be performed in any order. For example, a diagnostic catheter, not preloaded with a guide wire, can be advanced to the location of a vulnerable plaque lesion. A guide wire can then be advanced through or along the diagnostic catheter to the location of the vulnerable plaque lesion. The diagnostic catheter can then be removed, followed by advancement of the shield deployment catheter over or along the guide wire to the site of the vulnerable plaque.

According to the invention, determining the location of a vulnerable plaque in a blood vessel of a patient can be performed by any method or combination of methods. For example, catheter-based systems and methods for diagnosing and locating vulnerable plaques can be used, such as those employing optical coherent tomography (“OCT”) imaging, temperature sensing for temperature differences characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated herein in its entirety. A method and system for detecting and localizing vulnerable plaques based on the detection of biomarkers is disclosed in U.S. Pat. No. 6,860,851, which is hereby incorporated by reference herein in its entirety. Time-resolved laser-induced fluorescence spectroscopy (TR-LIFS) may also be used to detect and locate vulnerable plaques. U.S. Pat. No. 6,272,376 teaches TR-LIFS methods for detecting lipid-rich vascular lesions and is hereby incorporated by reference herein in its entirety.

In one embodiment of the invention, the step of determining the location of a vulnerable plaque lesion is performed using a low resolution Raman spectroscopy system that includes: a catheter comprising an excitation fiber through which multi-mode radiation can propagate to irradiate a target region of a lumen; a multi-mode laser for irradiating the target region to produce a Raman spectrum consisting of scattered electromagnetic radiation; a low resolution dispersion element positioned to receive and separate the scattered radiation into different wavelength components; a detection array, optically aligned with the dispersion element for detecting at least some of the wavelength components of the scattered light; and a processor for processing data from the detector array to determine the presence or absence of a vulnerable plaque lesion.

In a related embodiment of the invention, the step of determining the location of a vulnerable plaque lesion is performed according to a method that includes the steps of: providing a diagnostic catheter comprising an excitation fiber through which multi-mode radiation can propagate, the excitation fiber having a first end optically coupled to a multi-mode laser, and a second end positioned in optical alignment with a light directing element to direct radiation to a site within a lumen of a blood vessel; inserting the catheter into the lumen; activating the multi-mode laser to irradiate the lumen to produce a Raman spectrum consisting of scattered electromagnetic radiation; collecting a portion of the scattered radiation; separating the collected radiation into different wavelength components using a low resolution dispersion element; detecting at least some of the wavelength components of the scattered light using a detection array; and processing the data from the detection array to detect the presence or absence of a vulnerable plaque lesion.

Angiography using a radiopaque and/or fluorescent dye, for example, as known in the art, may be performed before, during and/or after the step of determining the location of the vulnerable plaque, for example, to assist in positioning the vulnerable plaque shield and/or occlusion balloon in a subject blood vessel.

In a variation, the embodiment further includes the steps of: deflating the occlusion balloon; and withdrawing the catheter from the patient, without the vulnerable plaque shield, leaving the shield deployed in the patient. Optionally, before withdrawing the catheter from the patient, the occlusion balloon can be deflated, withdrawn to a location within the lumen of the deployed vulnerable plaque shield and reinflated to further expand and/or secure the shield. The occlusion balloon can then be deflated and the catheter can be completely withdrawn from the patient.

The vulnerable plaque shield can, for example, have an at least substantially tubular configuration or can be capable of forming an at least substantially tubular configuration in its deployed state so that the outer wall of the shield will contact the inner wall of the blood vessel in which it is deployed. The vulnerable plaque shield can, for example, be a lightweight prosthesis. Since the vulnerable plaque shield is lightweight, occluding the flow of blood with the occlusion balloon during deployment of the vulnerable plaque shield prevents the shield from being moved or dislodged by the flow of blood from the desired location of deployment. This facilitates the precise placement of the vulnerable plaque shield in the location of the vulnerable plaque lesion.

The occlusion balloon can be a low-pressure balloon, for example, a low-pressure, elastomeric balloon as known in the art. Alternatively, the occlusion balloon can be a high-pressure balloon. The occlusion balloon may be located at or near the distal end of the catheter. As known in the art, the lumen of the balloon may be connected to a lumen running the length of the catheter to an external proximal port of the catheter, into which fluid or gas can be applied to increase the internal pressure of the occlusion balloon.

Another embodiment of the invention provides a combination occlusion and shield deployment catheter for treating vulnerable plaque conditions, that includes: a distal end for insertion into a patient's body and a proximal end; a selectively deployable occlusion balloon capable of occluding blood flow in a blood vessel; and a selectively deployable vulnerable plaque shield, located proximally along the catheter with respect to the occlusion balloon.

The catheter may also include one or more structural elements or features for guiding the catheter over or along a guide wire. These can be of any sort such as, but not limited to, a channel, passageway or lumen extending along at least part of the length of the catheter and sized and configured to accommodate a guide wire, or a hole or loop located, for example, at or near the distal end of the catheter and sized and configured to accommodate a guide wire. The catheter may include at least one radiopaque marker or portion to aid in the positioning of the vulnerable plaque shield and/or occlusion balloon during angiography.

The vulnerable plaque shield may be a self-expanding shield, for example, a lightweight self-expanding prosthesis formed, for example, from a shape-memory (shape-recovery) material, such as a memory metal alloy, such as nitinol, or a shape-memory polymer or polymer blend. Any sort of deployment mechanism for deploying a self-expanding shield can be used. For example, it is known in the art to mount an unexpanded self-expanding tubular prosthesis circumferentially about the shaft of a deployment catheter, wherein the self-expanding stent is constrained in the unexpanded state by a circumferentially surrounding, movable sheath member that can be selectively withdrawn to release the self-expanding tubular prosthesis.

In another variation, the combination catheter includes a circumferential outer projection at or near the proximal edge of the occlusion balloon. The function of this projection or “protective wing” is to prevent or reduce the possibility that the occlusion balloon will snag the distal end of the vulnerable plaque shield when the catheter is withdrawn.

A self-expanding vulnerable plaque shield for use according to the invention may, for example, be formed by a plurality of interconnected microfilaments, such as described in U.S. Publication No. U.S. 2005/0038503 which is hereby incorporated by reference herein in its entirety. The self-expanding force of a filament-based self-expanding prosthesis can be due, in part, to a plurality of filaments coherently engaged together to form a tube shape, for example, by braiding, weaving, and/or knitting, so as to radially expand in diameter. The filaments may be composed of an elastic metal, polymer, or composite of both, such as nitinol, stainless steel, platinum, or elgiloy and may, for example, be about 12-25 microns in thickness. In the case of a metal-polymer composite, the polymer may include a pharmacological agent within the polymer structure. Polymer filaments or polymer components of filaments may be biostable or biodegradable (bioresorbable).

To achieve the self expanding properties of a filament-based self-expanding prosthesis, a variety of different combinations of filament diameters, filament components, and engaging styles may be used. Typically, a self expanding prosthesis is annealed on a stainless steel mandrel fixture, which at least partially determines the expanded diameter of the self expanding prosthesis. For example, nitinol may be processed at about 500 degrees Celsius for about 10-15 minutes with a mandrel of a desired diameter. In another example, stainless steel, elgiloy, or MP35n alloy may be processed at temperatures of about 1000 degrees Celsius for relatively longer periods, such as 2-4 hours. The resulting annealed device will exhibit a desired expansion force to a desired diameter (as primarily determined by the mandrel size).

Examples of the structural makeup of a filament-based self-expanding prosthesis are listed below. In this regard, these examples reflect primary structural parameters and do not specify a length dimension since these devices can be made to any desired length for an intended purpose.

### EXAMPLE 1

For example, 72 filaments made from 0.0009 inch nitinol wire may be braided with a plain braid setup to create a 90 degree braid angle, ultimately forming a tube with a 4 mm diameter and a pore size of about 250 microns (cell size about 0.049 mm²).

### EXAMPLE 2

In another example, 56 filaments made from 0.001 inch stainless steel wire may be braided with a plain braid setup to create a 90 degree braid angle, ultimately forming a tube of 4 mm in diameter with 340 micron pore size (cell size about 0.091 mm²) and having a higher outward radial force than the previous example.

### EXAMPLE 3

In yet another example, 52 filaments of 0.001 inch stainless steel wire and 4 filaments of 0.0015 inch platinum wire (for radiopacity) may be braided with a plain braid setup to create a 90 degree braid angle, ultimately forming a tube of 4 mm in diameter with about 340 micron pore size (cell size about 0.091 mm²) and having a radial force higher than the first example.

### EXAMPLE 4

In another example, 0.001 inch nitinol wire is knit on a 16 needle machine with a 4 mm bore head (defining a 4 mm tube diameter), ultimately creating a tube with 500 micron pore size (cell size about 0.196 mm²).

### EXAMPLE 5

In another example, 0.001 stainless steel wire is knit on a 16 needle machine with a 4 mm bore head (defining a 4 mm tube diameter), ultimately creating a tube with 500 micron pore size (cell size about 0.196 mm²).

### EXAMPLE 6

In another example, 50 filaments of 0.001 inch nitinol wire may be woven to form a tube of 60 picks per inch and 4 mm in diameter, ultimately creating a tube with 500 micron pore size (cell size about 0.196 mm²).

### EXAMPLE 7

In another example, a sputtered nitinol film tube 10-15 microns thick may be used, ultimately creating a tube with 20-40 micron pore size.

### EXAMPLE 8

In yet another example, a sputtered nitinol film tube 10-15 microns thick with micro pleats may be used, ultimately creating a tube with 20-50 micron pore size. These micro pleats (elongated crimps in the prosthesis body) may be positioned along the axis of the prosthesis for expansion of the diameter of the prosthesis. Additionally, the micro pleats may be positioned circumferentially around the prosthesis for expansion in length.

### EXAMPLE 9

In another example, a sputtered nitinol film tube 10-15 microns is laser-machined with a pattern of holes to create a prosthesis having a 20-50 micron pore size.

### EXAMPLE 10

In another example, a sputtered nitinol film tube 10-15 microns thick with a textured mandrel may be used, creating a folding film. Generally, with a prosthesis formed from a sputtered film, the sputtered film is sputtered directly onto a mandrel with a textured surface. The textured surface of the mandrel could be, for example, a cross-hatched pattern or a “waffle” type pattern. Either way, the pattern creates small spring zones in the device that will operate similar to the aforementioned micro-pleats and allow the device to flex and expand more readily.

If desired, the number of filaments may vary along the length of the self-expanding prosthesis in order to increase or decrease the expansion diameter and expansion force exerted by the self expanding prosthesis. Specifically, as the number of filaments increase within a section of the self-expanding prosthesis, the expansion diameter and radial expansion force both increase. This property may be used to form a filament-based self-expanding prosthesis that expands outwardly to a greater diameter than the center section, allowing for a tighter fit between the ends of the prosthesis and the blood vessel. Additionally, if desired, the radial force of a filament-based self-expanding prosthesis may be increased by including a few larger diameter filaments engaged with relatively smaller sized filaments. In this respect, the overall pore size of the self expanding prosthesis may be kept small, while the outward radial force is increased.

Any other sort of self-expanding vulnerable plaque shield that has the desired range of porosity or cell sizes can also be used according to the invention. For example, a composite shield including a lightweight, self-expanding, metallic or non-metallic, tubular framework covered, internally and/or externally, by a covering or “skin” having the desired porosity when the device is in an expanded state may also be used. Such a covering may, for example, be a woven and/or non-woven fabric or sheet. Such a fabric or sheet can, for example, be made of polyethyleneterephthalate (PET), polytetrafluoroethylene (PTFE), or polyurethane-carbonate (PUC). Coverings can also be deposited by electrospinning fibers on to self-expanding frameworks to form vulnerable plaque shields for use according to the invention. Suitable electrospinning processes and materials are described, for example, in U.S. Publication Nos. 2003/0195611, 2003/0211135, and 2004/0051201, each of which is hereby incorporated by reference herein in its entirety.

Still other embodiments of the invention utilize a balloon-expandable vulnerable plaque shield, rather than a self-expanding shield. One such embodiment provides a method for treating a vulnerable plaque condition in an animal or human patient, that includes the steps of: determining the location of a vulnerable plaque in a patient; advancing a guide wire through the vasculature of the patient to the location of the vulnerable plaque; advancing a combination occlusion and shield deployment catheter over/along the guide wire to the location of the vulnerable plaque, the catheter including an occlusion balloon for occluding blood flow and an unexpanded, balloon-expandable vulnerable plaque shield mounted circumferentially about a deployment balloon, the shield being located proximally along the catheter with respect to the occlusion balloon; deploying the occlusion balloon to occlude blood flow; and while blood flow is at least substantially occluded by the deployed occlusion balloon, deploying the vulnerable plaque shield by expanding the deployment balloon so that the shield at least partially, and preferably, at least substantially, covers the vulnerable plaque.

A variation of the embodiment further includes the steps of: deflating the deployment balloon; deflating the occlusion balloon; and withdrawing the catheter (without the vulnerable plaque shield) from the patient, leaving the vulnerable plaque shield deployed in the patient.

The balloon-expandable vulnerable plaque shield may be a lightweight balloon-expandable meshwork or framework, for example, one having an at least substantially tubular configuration. In one variation, the vulnerable plaque shield is balloon-expandable under low pressure and the deployment balloon is a low-pressure balloon. In a related variation, the step of deploying the shield deployment balloon includes asserting a pressure to the lumen of the balloon of less than 12 ATMS, for example less than 11 ATMS, or less than 10 ATMS, or less than 9 ATMS, or less than 8 ATMS, or less than 7 ATMS, or less than 6 ATMS, or less than 5 ATMS, or less than 4 ATMS, or less than 3 ATMS or less than 2 ATMS or less than 1 ATMS. In a further, related variation, the step of deploying the shield deployment balloon includes asserting a pressure to the lumen of the balloon in the range of greater-than-zero to 1 ATMS, or 1-3 ATMS, or 3-5 ATMS, or 5-7 ATMS, or 7-9 ATMS, or 9-11 ATMS.

The occlusion balloon can, for example, also be a low-pressure balloon, such as a low-pressure elastomeric balloon or it can be a high-pressure balloon. In another variation of the embodiment, each of the occlusion balloon and the deployment balloon is a low-pressure balloon.

A further embodiment of the invention provides a combination occlusion and shield deployment catheter for treating vulnerable plaque conditions, that includes: a distal end for insertion into a patient's body and a proximal end; a selectively deployable occlusion balloon capable of occluding blood flow in a blood vessel; a selectively balloon-expandable vulnerable plaque shield mounted circumferentially on a deployment balloon, the shield being located proximally along the catheter with respect to the occlusion balloon. The catheter may also include one or more structural elements or features for guiding the catheter over or along a guide wire such as, but not limited to, those described above. In another variation of the embodiment, the catheter includes at least one radiopaque marker or portion. The catheter may also include a protective wing, as described above.

In a further variation of the embodiment, the occlusion balloon is a low-pressure balloon, for example, a low-pressure elastomeric balloon. Alternatively, the occlusion balloon can be a high-pressure balloon. In another variation, each of the occlusion balloon and the deployment balloon is a low-pressure balloon.

In still another variation, the deployment balloon is a low-pressure balloon. In related variation, one or the other or both of the shield deployment balloon and the balloon deployable shield are expandable by asserting a pressure to the lumen of the balloon of less than 12 ATMS, for example less than 11 ATMS, or less than 10 ATMS, or less than 9 ATMS, or less than 8 ATMS, or less than 7 ATMS, or less than 6 ATMS, or less than 5 ATMS, or less than 4 ATMS, or less than 3 ATMS or less than 2 ATMS or less than 1 ATMS. In a further, related variation, one or the other or both of the shield deployment balloon and the balloon deployable shield are expandable by asserting a pressure to the lumen of the balloon in the range of greater-than-zero to 1 ATMS, or 1-3 ATMS, or 3-5 ATMS, or 5-7 ATMS, or 7-9 ATMS, or 9-11 ATMS. Those skilled in the art will appreciate that materials can be readily selected by type, thickness and/or gauge to provide balloon-expandable, tubular prostheses that are expandable by the recited low pressure ranges. The occlusion balloon may, for example, be composed of latex or at least one synthetic polymer such as polyurethane, silicone rubber, a block copolymer of styrene-ethylene-butylene-styrene (SEBS) or a polyethylene, such as polyethylene terephthalate. Various occlusion balloon designs and compositions are disclosed in: U.S. Publication No. 2004/0267196 and U.S. Pat. Nos. 6,554,795; 6,652,480; 6,475,185; 6,234,996; 5,320,604; 5,222,970; and 4,456,011, each of which is incorporated by reference herein in its entirety.

Any other sort of balloon-expandable, non-self-expanding vulnerable plaque shield that has the desired range of porosity may be used according to the invention. For example, filaments coherently engaged together to form a tube shape, for example, by braiding, weaving, and/or knitting, so as to allow for radial expansion in diameter in response to an outwardly directed force imparted by a balloon can be used. The filaments may be composed of a metal, for example, stainless steel or titanium, a polymer, or a composite of both and may, for example, be about 12-25 microns in thickness. In the case of a metal-polymer composite, the polymer may include a pharmacological agent within the polymer structure. Polymer filaments or polymer components of filaments may be biostable or biodegradable (bioresorbable).

A composite, balloon-expandable, non-self-expanding shield including a lightweight, balloon-expandable, metallic or non-metallic, tubular framework covered, internally and/or externally, by a covering or “skin” having the desired porosity when the device is in an expanded state may also be used. Such a covering may, for example, be a woven and/or non-woven fabric or sheet. Such a fabric or sheet can, for example, be made of polyethyleneterephthalate (PET), polytetrafluoroethylene (PTFE), polyurethane-carbonate (PUC), or a biodegradable polymer such as polylactic acid (PLA). Coverings can also be deposited by electrospinning fibers on to self-expanding frameworks to form vulnerable plaque shields for use according to the invention. Suitable electrospinning processes and materials are described, for example, in U.S. Publication Nos. U.S. 2003/0195611, U.S. 2003/0211135, and U.S. 2004/0051201, each of which is hereby incorporated by reference herein in its entirety.

### EXAMPLE 11

### Delivery System for a Self-Expanding VP Shield

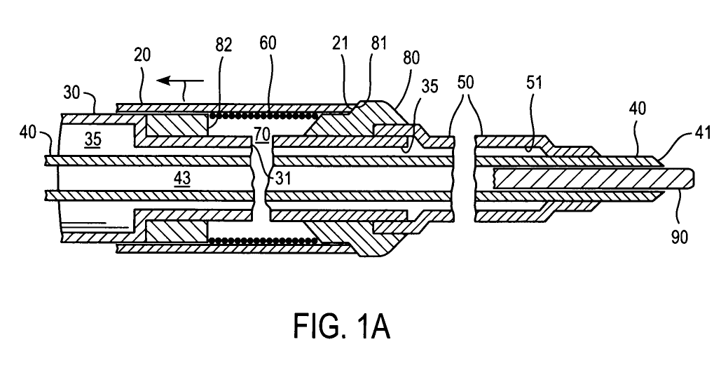

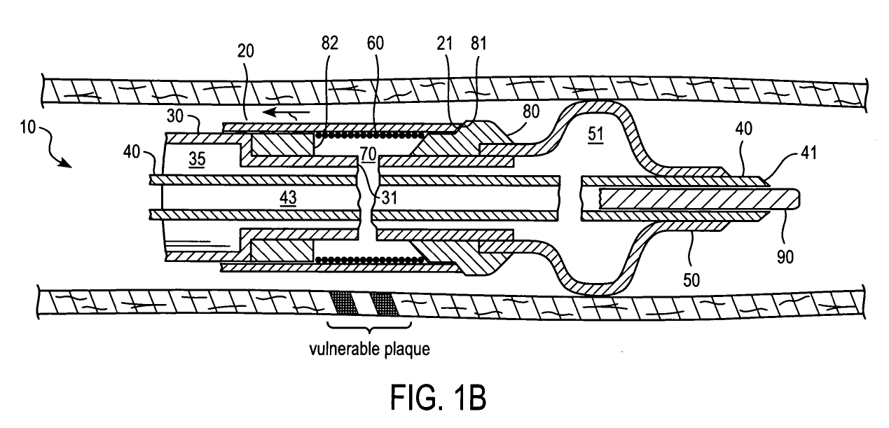

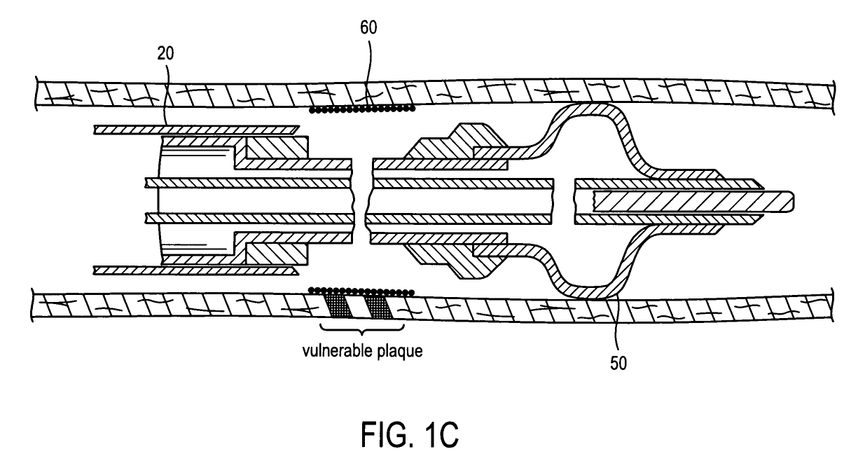

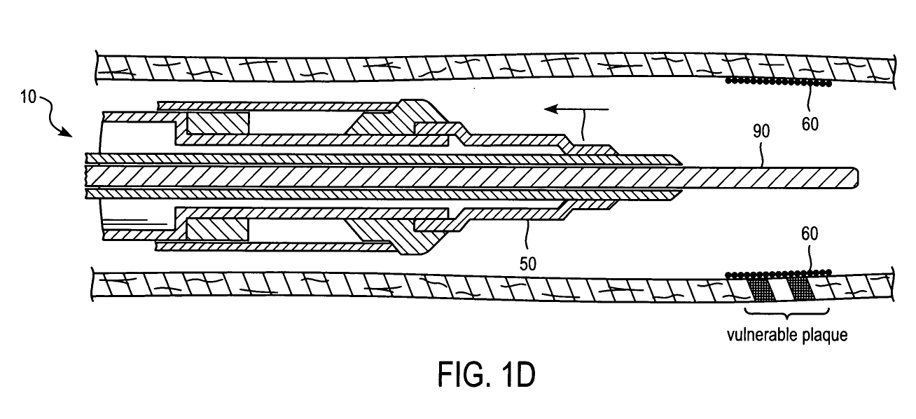

Example 11 relates to a catheter-based delivery system for a self-expanding vulnerable plaque shield and is described with reference to FIGS. 1A-1D. FIG. 1A shows, in cross-section, the distal portion of a vulnerable plaque shield delivery catheter 10 that includes: a moveable restraining sheath 20 that is slideably movable, as indicated by the arrow, over an outer tube 30; an inner tube 40; a low-pressure expandable occlusion balloon 50; a self-expanding vulnerable plaque shield 60 disposed (in an unexpanded state) in a vulnerable plaque shield containment cavity 70; and radiopaque marker bands 80 and 82 that serve as structural elements of the catheter. The moveable restraining sheath 20 has a tapered distal end 21 designed to join onto the conical surface 81 of the distal marker band 80 thus forming a smooth and continuous outer surface for the distal portion of the catheter 10. As shown in this example, the very low pressure occlusion balloon 50 in its unexpanded state is recessed with respect to the outer dimension of outer tube 30. The maximum outer dimension of marker band 80 exceeds the outer dimension of outer tube 30.

The outer tube 30 has a distal portion 31 that is adhesively bonded near its distal end to both the proximal end of the balloon 50 and distal marker band 80. The distal end of the balloon 50 can be adhesively joined to the outer surface of the tube 40 near its tapered distal end 41.

Lumen 43 of inner tube 40 serves as a passageway for a guide wire 90 that enters from the proximal end of the catheter. The illustrated embodiment is an example of an over-the-wire configuration. Lumen 35 formed between inner tube 40 and outer tube 30 is used to deliver positive and negative pressure from the proximal end of the catheter to the balloon chamber 51 to expand and deflate the very low pressure occlusion balloon 50.

The catheter may be designed to have any desired linear distance between the distal end of the vulnerable plaque shield containment cavity 70 and the proximal end of the very low pressure occlusion balloon 50. The catheter may also be designed to have a containment cavity 70 of any desired axial length, as indicated by the illustrated interruption of the catheter elements located centrally of shield 60, in order to accommodate a self-expanding vulnerable plaque shield 60 of any desired axial length. For example, a vulnerable plaque shield having a length in the range of 1.0-4.0 cm may be used with a suitably sized delivery catheter. Similarly, low-pressure occlusion balloon 51 can be of any axial length, as indicated by the illustrated interruption of catheter elements in the balloon region of the catheter.

FIG. 1B shows the delivery catheter of FIG. 1A positioned in a blood vessel, such as an artery, for the delivery of a vulnerable plaque shield. As shown, the catheter 10 is positioned so that the vulnerable plaque shield 60 is aligned with a vulnerable plaque lesion to be treated, indicated schematically by the hatched areas. The low-pressure occlusion balloon 50 has been inflated and is in contact with the vessel wall in order to block the flow of blood.

FIG. 1C shows that moveable sheath 20 has been fully retracted to release vulnerable plaque shield 60, which is shown in its expanded state in contact with the vessel wall and covering the vulnerable plaque. In practice, moveable sheath 20 may be slowly retracted or retracted in steps to permit the vulnerable plaque shield 60 to progressively expand into contact with the vessel wall from its distal end to its proximal end. Such a procedure aids in the precise positioning of the vulnerable plaque shield 60. However, a rapid retraction of moveable sheath 20 to rapidly deploy vulnerable plaque shield 60 is also within the scope of the invention.

FIG. 1D shows the vulnerable plaque shield 60 deployed over the vulnerable plaque of the blood vessel while the deployment catheter 10 has begun to be withdrawn from the patient. The catheter 10 may, for example, be withdrawn over guide wire 90 from the patient followed by withdrawal of the guide wire itself. The deployed vulnerable plaque shield 60 provides at least some embolic protection against rupture of the vulnerable plaque and passivates the vulnerable plaque through one or more processes such as growth and/or migration of endothelial cells to cover the lumen-side wall of the vulnerable plaque shield (endothelialization).

### EXAMPLE 12

### Method of Using a Self-Expanding VP Shield

Example 12 relates to a method of treating a vulnerable plaque condition in a human artery using a catheter-based delivery system for a vulnerable plaque shield (DSVPS). The DSVPS consists of an intravascular catheter (IC) that has a very low pressure, conformable, inflatable balloon (VPB) attached or built-in circumferentially around the IC on the distal portion of the IC, which may be inflated from a proximal inflation port outside the human body. Proximal to the VPB, a self-expanding vulnerable plaque shield (VPS) is positioned circumferentially around the IC.

A small protective wing (PW) may be provided adjacent to the proximal edge of the VPB. A movable restraining sheath (MRS) is placed circumferentially extending the length of the DSVPS to cover and thereby restrain the vulnerable plaque shield (VPS) in an unexpanded state. The MRS may be controlled and withdrawn from its original position from outside the human body. After appropriate packaging and sterilization, the final, fully assembled DSVPS is delivered to the human artery over a previously placed guide-wire by a trained physician. Fluoroscopy may be used to follow the positioning of the DSVPS, using radiopaque markers provided on the MRS and the IC, until the VPS is positioned precisely at the location of a previously detected and located vulnerable plaque lesion. The VPB is inflated using a very low pressure, for example, 1 or 2 ATMS or under 30 psi (versus normal balloon pressure for an angioplasty balloon and or a stent delivery balloon, which typically is 12 to 16 ATMS or 180 to 240 psi). This inflation of the VPS occludes the blood flow in the human artery, at least substantially preventing blood flow past the inflated balloon. The MRS is then progressively withdrawn to a point just proximal to the proximal edge of the VPS. This progressively releases the VPS to fully expand, thus providing a protective covering over at least substantially all of the vulnerable plaque lesion.

After the expansion of the VPS, the physician deflates the VPB and optionally pulls back the IC to position the deflated VPB within the expanded VPS. The PW is provided to prevent the proximal end of the VPB from snagging the distal end of the VPS, but the PW may not be needed if the material for the VPB closely “hugs” the IC shaft in the deflated state. After alignment of the VPB within the lumen of the VPS, the physician then re-inflates the VPB to a very low pressure, which will gently position the VPS to be fully apposed to the lumen of the artery, assuring full coverage of the vulnerable plaque lesion.

Design features known in the art can be readily adapted for implementing certain aspects of the methods and systems of the present invention. U.S. Pat. No. 5,743,874, which is hereby incorporated by reference herein in its entirety, discloses an integrated catheter for balloon angioplasty and stent delivery that includes a distal balloon for pre-dilatation of a vascular stenosis and a more proximally located, sheath-releasable, self-expanding stent. U.S. Pat. No. 6,231,588, which is hereby incorporated by reference herein in its entirety, discloses a low profile catheter for angioplasty and occlusion that includes a distal occlusion balloon and a more proximally located angioplasty balloon.

The invention has been described herein with respect to various examples and embodiments. However, it should be understood that still other variations within the scope and spirit of the invention may be apparent to those skilled in the art. Accordingly, the scope of the invention should be determined with respect to the appended claims and the full scope of equivalents to which they are entitled.

What is claimed is:

1. A method of treating a vulnerable plaque condition in a patient, comprising the steps of:
determining the location of a vulnerable plaque in a patient;
advancing a combination occlusion and shield deployment catheter to the location of the vulnerable plaque, wherein the catheter comprises:
an occlusion balloon; and
an unexpanded, self-expanding vulnerable plaque shield, the shield being located proximally along the catheter with respect to the occlusion balloon;
deploying the occlusion balloon to occlude blood flow; and
while blood flow is at least substantially occluded by the deployed occlusion balloon, deploying the self-expanding vulnerable plaque shield so that it at least partially covers the vulnerable plaque.

2. The method of claim 1, further comprising the step of:
advancing a guide wire through the vasculature of the patient to the location of the vulnerable plaque; and wherein
the step of advancing the combination occlusion and shield deployment catheter to the location of the vulnerable plaque comprises advancing the catheter along the guide wire to the location of the vulnerable plaque.

3. The method of claim 1, further comprising the steps of:
deflating the occlusion balloon; and
withdrawing the catheter from the patient, leaving the vulnerable plaque shield deployed in the patient.

4. The method of claim 1, wherein the vulnerable plaque shield is an at least substantially tubular, filament-based prosthesis.

5. The method of claim 1, wherein the occlusion balloon is a low-pressure balloon.

6. A combination occlusion and shield deployment catheter for treating vulnerable plaque conditions, comprising:
a distal end for insertion into a patient's body and a proximal end;
a selectively deployable occlusion balloon capable of occluding blood flow in a blood vessel;
a selectively deployable, vulnerable plaque shield, located proximally along the catheter with respect to the occlusion balloon.

7. The catheter of claim 6, wherein the vulnerable plaque shield has a shield wall having cell sizes at least predominantly in the range of 0.038 mm²-0.180 mm².

8. The catheter of claim 7, wherein the shield wall of the vulnerable plaque shield is an at least substantially braided structure.

9. The catheter of claim 6, wherein the vulnerable plaque shield has a shield wall having average cell sizes in the range of about 0.038 mm² to about 0.180 mm².

10. The catheter of claim 9, wherein the shield wall of the vulnerable plaque shield is at least substantially a braided structure.

11. The catheter of claim 6, further comprising at least one radiopaque marker or portion.

12. The catheter of claim 6, wherein the occlusion balloon is a low-pressure balloon.

13. The catheter of claim 6, wherein the shield is self-expanding.

14. The catheter of claim 13, wherein the shield is an at least substantially tubular, filament-based prosthesis.

15. The catheter of claim 6, further comprising a protective wing located at or near the proximal edge of the occlusion balloon.

16. A method of treating a vulnerable plaque condition in a patient, comprising the steps of:
determining the location of a vulnerable plaque in a patient using a low-resolution Raman spectroscopy system;
advancing a combination occlusion and shield deployment catheter to the location of the vulnerable plaque, wherein the catheter comprises:
an occlusion balloon; and
an unexpanded, self-expanding vulnerable plaque shield, the shield being located proximally along the catheter with respect to the occlusion balloon;
deploying the occlusion balloon to occlude blood flow; and
while blood flow is at least substantially occluded by the deployed occlusion balloon, deploying the self-expanding vulnerable plaque shield so that it at least partially covers the vulnerable plaque.

17. The method of claim 16, further comprising the steps of:
deflating the occlusion balloon; and
withdrawing the catheter from the patient, leaving the vulnerable plaque shield deployed in the patient.

18. A method of treating a vulnerable plaque condition in a patient, comprising the following steps:
a step for determining the location of a vulnerable plaque in a patient;
advancing a combination occlusion and shield deployment catheter to the location of the vulnerable plaque, wherein the catheter comprises:
an occlusion balloon; and
an unexpanded, self-expanding vulnerable plaque shield, the shield being located proximally along the catheter with respect to the occlusion balloon;
deploying the occlusion balloon to occlude blood flow; and
while blood flow is at least substantially occluded by the deployed occlusion balloon, deploying the self-expanding vulnerable plaque shield so that it at least partially covers the vulnerable plaque.

19. A method of treating a vulnerable plaque condition in a patient, comprising the steps of:
determining the location of a vulnerable plaque in a patient;
advancing a combination occlusion and shield deployment catheter to the location of the vulnerable plaque, wherein the catheter comprises:
an occlusion balloon; and
an unexpanded, balloon-expandable vulnerable plaque shield mounted circumferentially about a deployment balloon, the shield being located proximally along the catheter with respect to the occlusion balloon;
deploying the occlusion balloon to occlude blood flow;
while blood flow is at least substantially occluded by the deployed occlusion balloon, deploying the vulnerable plaque shield by expanding the deployment balloon so that the shield at least partially covers the vulnerable plaque.

20. The method of claim 19, further comprising the step of:
advancing a guide wire through the vasculature of the patient to the location of the vulnerable plaque; and wherein
the step of advancing the combination occlusion and shield deployment catheter to the location of the vulnerable plaque comprises advancing the catheter along the guide wire to the location of the vulnerable plaque.

21. The method of claim 19, further comprising the steps of:
deflating the deployment balloon;
deflating the occlusion balloon; and
withdrawing the catheter from the patient, leaving the vulnerable plaque shield deployed in the patient.

22. The method of claim 19, wherein the vulnerable plaque shield is an at least substantially tubular, filament-based prosthesis.

23. The method of claim 19, wherein the occlusion balloon is a low-pressure balloon.

24. The method of claim 19, wherein the deployment balloon is a low-pressure balloon.

25. The method of claim 19, wherein each of the occlusion balloon and the deployment balloon is a low-pressure balloon.

26. A combination occlusion and shield deployment catheter for treating vulnerable plaque conditions, comprising:
a distal end for insertion into a patient's body and a proximal end;
a selectively deployable occlusion balloon capable of occluding blood flow in a blood vessel;
a selectively balloon-expandable vulnerable plaque shield mounted circumferentially on a deployment balloon, the shield being located proximally along the catheter with respect to the occlusion balloon.

27. The catheter of claim 26, wherein the vulnerable plaque shield has a shield wall having cell sizes at least predominantly in the range of 0.038 mm²-0.180 mm².

28. The catheter of claim 26, wherein the shield wall of the vulnerable plaque shield is at least substantially a braided structure.

29. The catheter of claim 26, wherein the vulnerable plaque shield has a shield wall having average cell sizes in the range of about 0.038 mm² to about 0.180 mm².

30. The catheter of claim 29, wherein the shield wall of the vulnerable plaque shield is a braided structure.

31. The catheter of claim 26, further comprising at least one radiopaque marker or portion.

32. The catheter of claim 26, wherein the occlusion balloon is a low-pressure balloon.

33. The catheter of claim 26, wherein the deployment balloon is a low-pressure balloon.

34. The catheter of claim 26, wherein each of the occlusion balloon and the deployment balloon is a low-pressure balloon.

35. The catheter of claim 26, further comprising a protective wing located at or near the proximal edge of the occlusion balloon.
